(19) United States
(12) Patent Application Publication
(10) Pub. No.: US 2003/0013985 A1
(43) Pub. Date: Jan. 16, 2003

(54) Method for sensing temperature profile of a hollow body organ

(76) Inventor: Vahid Saadat, Saratoga, CA (US)

Correspondence Address: JOHNEY U. HAN, MORRISON & FOERSTER LLP, 755 PAGE MILL ROAD, PALO ALTO, CA, 94304-1018, US

(21) Appl. No.: 09/904,080

(22) Filed: Jul. 12, 2001

(51) Int. Cl. A61B 5/00; A61M 25/00; A61B 5/103; A61B 5/117

(52) U.S. Cl. 600/549; 600/585; 600/587

(57) ABSTRACT

A method for sensing the temperature profile of a hollow body organ utilizes a catheter and a hollow guidewire. The guidewire is configured as a plurality of helical loops of greater diameter than the catheter when unconstrained. When constrained within the catheter, the guidewire can be advanced to a region of interest in hollow body organ. The catheter can be withdrawn, leaving the guidewire in place in an expanded configuration wherein the helical loops contact the inner wall of the hollow body organ. A temperature sensor is moveable within the guidewire to sense the temperature at multiple locations.

### TECHNICAL FIELD OF THE INVENTION

This invention relates generally to invasive medical devices and more particularly to methods using such devices for sensing the temperature of the interior wall of a hollow body organ such as a blood vessel.

### BACKGROUND OF THE INVENTION

Acute ischemic syndromes involving arterial blood vessels, such as myocardial infarction, or heart attack, and stroke, frequently occur when atherosclerotic plaque ruptures, triggering the formation of blood clots, or thrombosis. Plaque that is inflamed is particularly unstable and vulnerable to disruption, with potentially devastating consequences. Therefore, there is a strong need to detect and locate this type of plaque so that treatment can be initiated before the plaque undergoes disruption and induces subsequent life-threatening clotting.

Various procedures are known for detecting and locating plaque in a blood vessel. Angiography is one such procedure in which X-ray images of blood vessels are generated after a radiopaque dye is injected into the blood stream. This procedure is capable of locating plaque in an artery, but is not capable of revealing whether the plaque is the inflamed, unstable type.

Researchers, acting on the theory that inflammation is a factor in the development of atherosclerosis, have discovered that local variations of temperature along arterial walls can indicate the presence of inflamed plaque. The temperature at the site of inflamation, i.e., the unstable plaque, is elevated relative to adjacent plaque-free arterial walls.

Using a tiny thermal sensor at the end of a catheter, the temperature at multiple locations along an arterial wall were measured in people with and without atherosclerotic arteries. In people free of heart disease, the temperature was substantially homogeneous wherever measured: an average of 0.65 degrees F. above the oral temperature. In people with stable angina, the temperature of their plaques averaged 0.19 degrees F. above the temperature of their unaffected artery walls. The average temperature increase in people with unstable angina was 1.23 degrees F. The increase was 2.65 degrees F. in people who had just suffered a heart attack. Furthermore, temperature variation at different points at the plaque site itself was found to be greatest in people who had just had a heart attack. There was progressively less variation in people with unstable angina and stable angina.

The temperature heterogeneity discussed above can be exploited to detect and locate inflamed, unstable plaque through the use of cavity wall profiling apparatus. Typically, cavity wall profiling apparatus are comprised of temperature indicating probes such as thermocouples, thermistors, fluorescence lifetime measurement systems, resistance thermal devices and infrared measurement devices.

One problem with conventional cavity wall profiling apparatus is that they usually exert an undue amount of force on the region of interest. If the region of interest cannot withstand these forces, it may be damaged. The inside walls of a healthy human artery are vulnerable to such damage. Furthermore, if inflamed, unstable plaque is present it may be ruptured by such forces.

Another problem with conventional cavity wall profiling apparatus is that they can only measure the temperature at one specific location. In order to generate a map of the cavity temperature variation, one would need to move the temperature indicating probe from location to location. This can be very tedious, can increase the risk of damaging the vessel wall or rupturing vulnerable plaque, and may not resolve temporal characteristics of the profile with sufficient resolution. An array of probes could be employed but that could be very big and heavy.

### SUMMARY OF THE INVENTION

According to one aspect of the invention, a device is provided for sensing the temperature profile of a hollow body organ. The device includes a catheter, a hollow guidewire, and a temperature sensor longitudinally moveable within the guidewire. The guidewire has an expanded configuration externally of the catheter including a plurality of helical loops of greater diameter than the catheter. The guidewire also has a contracted configuration internally of the catheter and is of a lesser diameter than the catheter.

According to another aspect of the invention, the device is used by contracting the guidewire elastically and constraining the guidewire within the catheter. The catheter and guidewire are advanced to a region of interest in a hollow body organ. The catheter is withdrawn while securing the guidewire against substantial longitudinal movement relative to the hollow body organ, resulting in the guidewire self-expanding into helical loops in contact with the hollow body organ. As the temperature probe is advanced to a region of interest, the hollow guidewire and the probe remain within the catheter. The temperature sensing is done while the hollow guidewire is deployed out of the catheter and the temperature probe is retracted within the hollow guidewire. The temperature probe is moved through the guidewire to sense the temperature of the hollow body organ at multiple locations.

Further aspects and advantages of the present invention are apparent from the following description of a preferred embodiment referring to the drawings.

### BRIEF DESCRIPTION OF THE DRAWINGS

In the drawings,

FIG. 1 is a perspective, partially cut-away view of an arterial hollow body organ in which a preferred embodiment of the present invention is deployed;

FIG. 2 is an enlarged perspective view of the embodiment of FIG. 1;

FIG. 3 is an enlarged perspective view of another preferred embodiment of the present invention;

FIG. 4 is an enlarged perspective view of a further preferred embodiment of the present invention;

FIG. 5 is a block diagram of a controller useful in connection with the embodiment of FIG. 4; and

FIG. 6 is a perspective view, partially is section, of yet another preferred embodiment of the present invention.

### DESCRIPTION OF THE PREFERRED EMBODIMENT

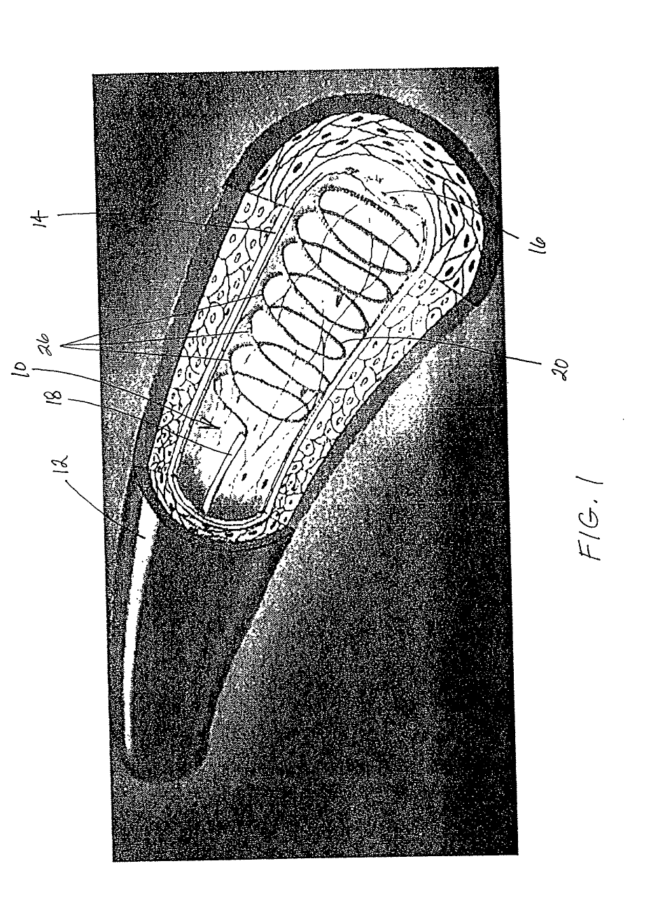

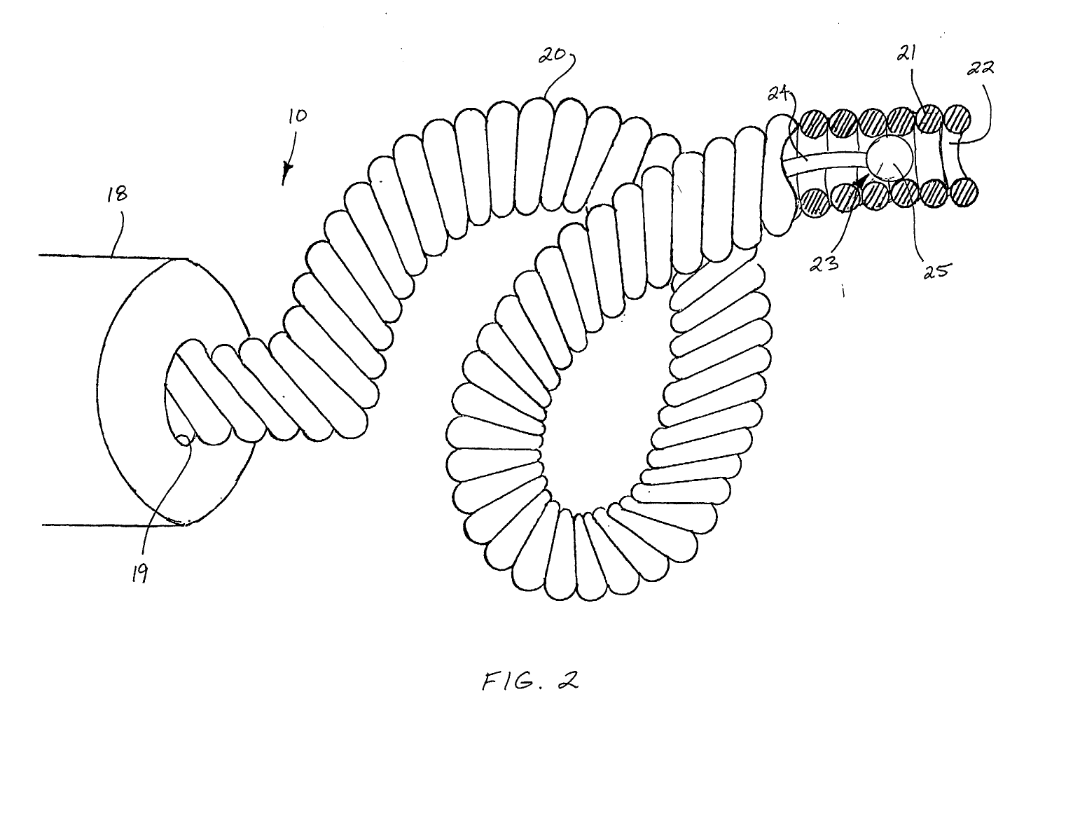

FIGS. 1 and 2 show an expandable device 10 for profiling the wall of a hollow body organ. Device 10 is shown deployed in a hollow body organ comprising an arterial blood vessel 12 having an endothelium 14 forming the inner wall thereof. A plaque 16 is disposed in endothelium 14.

Device 10 includes a lumened catheter 18 having a central lumen 19, a hollow guidewire 20 comprising a tubular helix formed of metal wire 21 or the like in the shape of a coil defining a central lumen 22, and a temperature probe 23 disposed within the lumen 22 of guidewire 20. The temperature probe 23 comprises a flexible elongate member 24 of sufficient stiffness to permit insertion into and withdrawal from lumen 22 of guidewire 20, following the curves thereof, without bending or kinking. A thermal sensor 25 is disposed at the distal end of the temperature probe 23, and conventional conductors or other signal carrying structures (not shown) are provided to convey signals from the thermal sensor along the guidewire 20 and out of the proximal end of guidewire 20 for connection to appropriate signal processing apparatus that converts the signals to a temperature indication. Thermal sensor 25 can be a thermocouple or a thermistor, for example.

The temperature probe can be made of metal wire, or a suitable plastic material, or a combination of both such as a metal wire coated with lubricous polymer material such as polytetrafluoroethylene (PTFE or Teflon®), polyethylene or other lubricous polymer material as known in the art. The coils of guidewire 20 may also be coated with a lubricous polymer such as PTFE to aid the insertion and withdrawal of the temperature probe within the lumen of guidewire 20. Such a coating also helps to thermally isolate the adjacent coils from one another and make the thermal mapping more precise. In other words, it will reduce the spread of heat from a hot zone to a normothermic zone.

Guidewire 20 is made of thin wire 21 wound, for example around a mandrel, into small helical coils of desired diameter that lie tightly adjacent one another to form a hollow tube having a central passageway or lumen 22 therethrough. Guidewire 20 has an outer diameter somewhat less than the inner diameter of catheter 18 to permit guidewire 20 to slide freely within the lumen 19 of catheter 18. In addition, guidewire 20, in its relaxed configuration, is shaped as large, loosely spaced helical loops 26. Guidewire 20 can be deformed from this relaxed configuration under force, and when the force is removed guidewire 20 returns to the relaxed, looped configuration.

Temperature probe 23 has a stiffness substantially less than that of the guidewire 20 and has flexibility while having excellent pushability. Flexibility permits temperature probe 23 to follow the curves of helical loops 26 of guidewire 20 without forcing guidewire 20 to become straight.

The self-looping characteristic of guidewire 20 can be accomplished in several ways. One way is to construct guidewire 20 of spring steel that can be deformed into a relatively straight configuration when withdrawn into catheter 18, but which springs back to its looped configuration when extruded from catheter 18 and released from constraint. Another way is to construct guidewire 20 of superelastic nitinol and take advantage of the martensitic transformation properties of nitinol. Guidewire 20 can be inserted into catheter 18 in its straight form and kept cool within the catheter by the injection of cold saline through catheter 18 and over guidewire 20. Upon release of guidewire 20 into the bloodstream, it will warm up and change to its austenite memory shape based on the well-known martensitic transformation by application of heat and putting the material through its transformation temperature.

Guidewire 20 can also be made out of a composite such as a nitinol tube within the guidewire structure. In this fashion, the martensitic or superelastic properties of nitinol can be combined with the spring steel characteristics of the spring and lead to a desirable composition. Other suitable materials for guidewire 20 include copper, constantan, chromel or alumel.

In use, the procedure is to first advance the catheter, separately, or together with the hollow guidewire and the temperature probe therewithin, to the region of interest. Thereafter the hollow guidewire and the temperature probe are deployed beyond the distal end of the catheter. At this time the temperature probe can be positioned to a desired longitudinal location within the guidewire, preferably so that the tip of the probe is at the distal end of the deployed guidewire. Preferably, the temperature probe is inserted into the lumen 22 of guidewire 20 from the proximal end until the tip with the thermal sensor 25 is disposed at the distal end of guidewire 20. Guidewire 20 is inserted into the lumen 19 of catheter 18 from the proximal end, thereby constraining guidewire 20 into a substantially straight configuration. Using conventional percutaneous insertion techniques, access to the blood vessel 12 is obtained surgically and device 10 is advanced through the blood vessel 12 to the region of interest.

To deploy the probe, guidewire 20 is secured against movement relative to the patient, catheter 18 is slowly withdrawn such that guidewire 20 emerges from the distal end of catheter 20 and reverts to its looped configuration within the blood vessel 12. Guidewire 20 remains substantially fixed in the axial direction relative to the blood vessel 12 as catheter 18 is withdrawn, with the reformed loops 26 springing radially outwardly into contact with the vessel wall 14. The relative lack of movement between guidewire 20 and vessel wall 14 alleviates the risk of damage to vessel wall 14 and the risk of rupturing unstable plaque.

With guidewire 20 exposed and lying in helical contact with the wall 14 of blood vessel 12, the temperature probe 23 is able to sense the localized temperature of the vessel wall 14 through the guidewire 20 at the region where the thermal sensor 25 is located. By slowly withdrawing the temperature probe 23 from guidewire 20, the thermal sensor 25 traverses a helical path around the wall 14 of the blood vessel 12, permitting temperature measurements to be taken at intervals of different regions of the vessel wall 14. By withdrawing the temperature probe 23 at a constant rate, the location of the thermal sensor 25 relative to the distal end of the guidewire 20 can be determined as a function of time, so that a temperature profile of the blood vessel 12 can be mapped.

Once the mapping is completed, the catheter 18 can be pushed forward again while securing guidewire 20 against longitudinal movement. Catheter 18 will thereby re-sheath guidewire 20 and constrain it in a substantially straight configuration for withdrawal from the blood vessel 12 so that the temperature probe will be able to advance to the forward position.

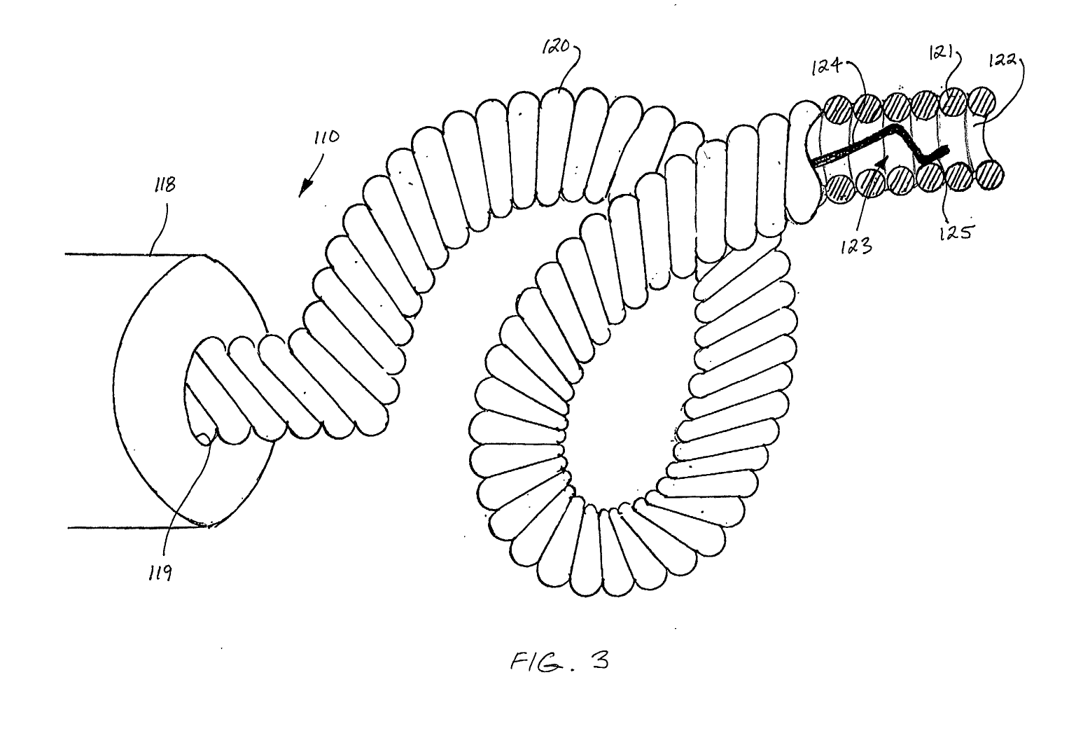

FIG. 3 shows a second preferred embodiment of an expandable device 110 for profiling the wall of a hollow body organ. Device 110 can be deployed in a hollow body organ in a manner similar to that shown in FIG. 1 and described above with respect to the first embodiment of expandable device 10. Components of device 110 that are similar in structure and function to corresponding components of device 10 of FIG. 1 are designated by like reference numerals in the 100 series but having the same last two digits. The description of device 10 above applies also to device 110 unless described otherwise below.

Device 110 includes a lumened catheter 118, a hollow guidewire 120, and a temperature probe 123 disposed within the lumen 122 of guidewire 120. The temperature probe 123 comprises a flexible elongate member 124 of sufficient stiffness to permit insertion into and withdrawal from lumen 122 of guidewire 120, following the curves thereof, without bending or kinking. A thermal sensor 125 is disposed at the distal end of the temperature probe 123, sensor 125 comprising a dog-leg bend at the distal end of elongate member 124 of sufficient length and angular orientation to remain in contact with the interior surface of lumen 122 of guidewire 120 as temperature probe 123 is moved axially within guidewire 120.

Guidewire 120 and thermal sensor 125 are composed of dissimilar metals such that contact therebetween forms a thermocouple junction that generates an electrical voltage proportional to the temperature of the thermocouple junction. Elongate member 124 of temperature probe 123 comprises one conductor and guidewire 120 comprises another conductor of the resulting thermocouple for conveying signals from the thermal sensor 125 to the proximal end of guidewire 120 for connection to appropriate signal processing apparatus that converts the signals to a temperature indication. Suitable materials for guidewire 120 and thermal sensor 125 to create a thermocouple include copper, constantan, chromel, alumel, and the like, the lead serving as the thermal sensor 125 being suitably insulated except at the tip thereof.

Device 110 of FIG. 3 can be used in a manner substantially similar to the manner of use described above with respect to device 10 of FIG. 1.

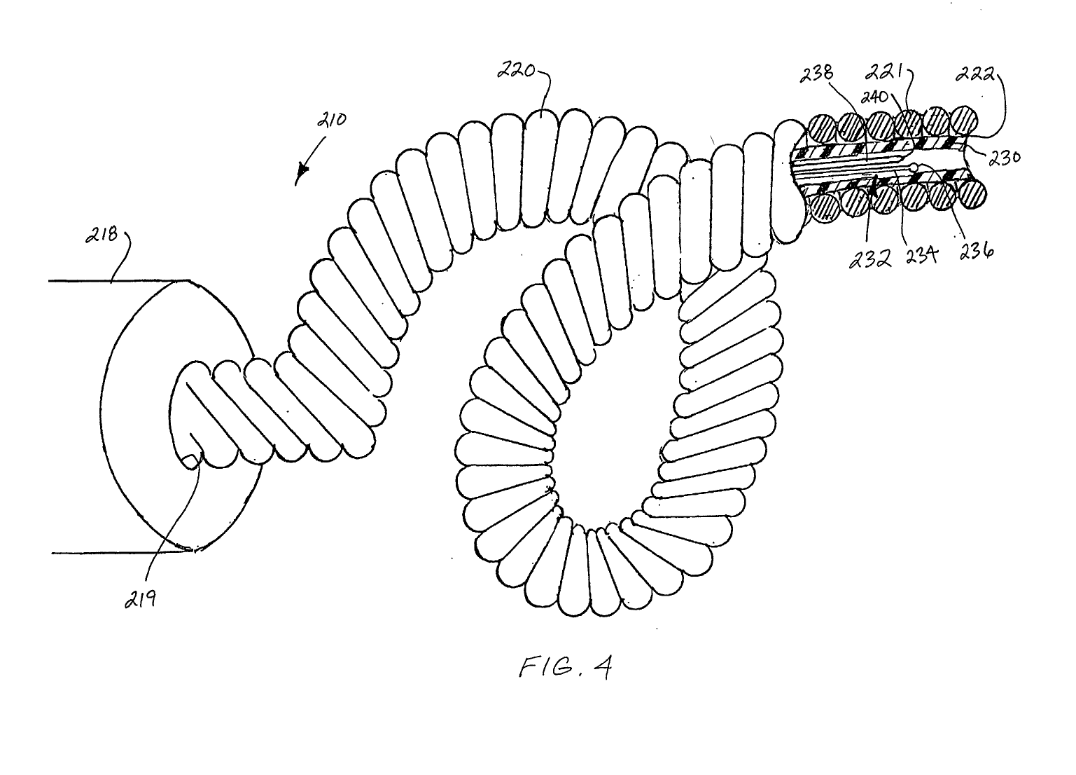

FIG. 4 shows yet another preferred embodiment of an expandable device 210 for profiling the wall temperature of a hollow body organ. Device 210 can be deployed in a hollow body organ in the manner shown in FIG. 1 and described above with respect to the first embodiment of expandable device 10. Components of device 210 that are similar in structure and function to corresponding components of device 10 of FIG. 1 are designated by like reference numerals in the 200 series but having the same last two digits. The description of device 10 above applies also to device 210 unless described otherwise below.

Device 210 includes a lumened catheter 118 and a hollow guidewire 120. The inner surface of lumen 222 of guidewire 220 is lined with a thermochromic material 230 that is sensitive to a change of temperature of the guidewire 220. The color of the thermochromic material 230 varies as a function of temperature.

Disposed within lumen 222 of guidewire 220, inwardly of thermochromic material 230, is an optical probe 232 including an illuminating optical fiber 234 having a radially emitting diffuser 236 at the distal end thereof, and a sensing optical fiber 238 having a conically beveled distal end 240 for collecting light. Optical fibers 234 and 238 are moveable in unison within lumen 222 in a manner similar to that of temperature probes 23 and 123 described above with reference to FIGS. 1-3. An illuminating electromagnetic radiation source is connected to the proximal end of illuminating optical fiber 234 provides illuminating radiation that is guided by optical fiber 234 to the region of interest within the hollow body organ, and diffused radially by diffuser 236 to illuminate the interior of lumen 222, particularly thermochromic material 230. The illuminating radiation can be in the visible, infrared or ultraviolet portions of the spectrum. Radiation from diffuser 236 is differentially absorbed and reflected by thermochromic material 230, according to the color of material 230 which is indicative of the temperature of guidewire 220 in contact with the wall of the hollow body organ in the region of interest.

The light reflected from thermochromic material 230, having wavelengths indicative of the color thereof, is collected by distal end 240 and directed toward the proximal end of sensing optical fiber 238. An appropriate optical reflectance spectrometry device connected to the proximal end of sensing optical fiber 238 generates an electrical signal indicative of the color, and therefore temperature, of thermochromic material 230.

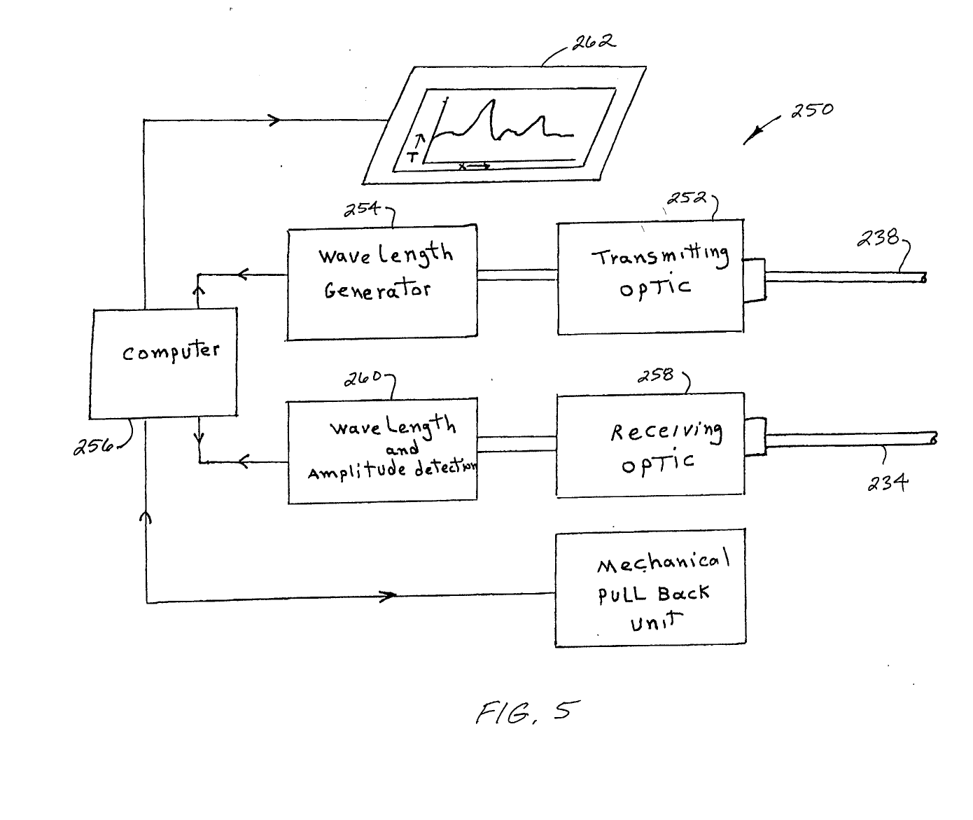

FIG. 5 shows a block diagram of a control device 250 suitable for use with device 210 of FIG. 4. An optical transmitter 252 generates light for transmission through optical fiber 238 as discussed above. Transmitter 252 is operably connected to a wavelength generator 254 that generates signals indicative of the wavelength of the light transmitted by transmitter 252, which signal is conveyed as an input to a computer 256. An optical receiver 258 receives light reflected from thermochromic material 230 (FIG. 4) through optical fiber 234 as discussed above. Receiver 258 is operably connected to a wavelength and amplitude detector 260 that generates signals indicative of the wavelength and amplitude of the light received by receiver 258, which signals are conveyed as an input to a computer 256. A processed output signal from computer 256 generates a graphical display 262 of detected color, i.e., temperature, as a function of linear displacement of optical probe 232 relative to catheter 218. A mechanical pull-back device 264 is mechanically connected to optical probe 232 and is controlled by and sends feedback signals to computer 256, which signals contribute to the generation of the display 262.

Device 210 of FIG. 4 can be used in a manner substantially similar to the manner of use described above with respect to device 10 of FIG. 1.

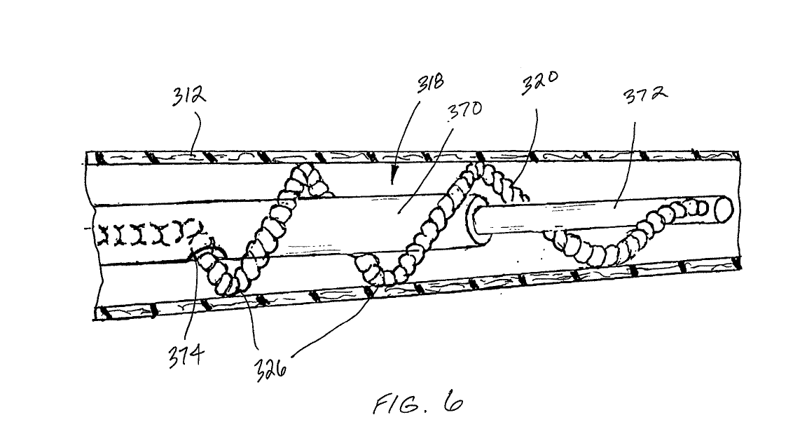

FIG. 6 shows still another preferred embodiment of the present invention that can incorporate any of the various temperature sensing technologies described above with respect to the first, second and third embodiments. Catheter 318 includes a first portion 370 and a second portion 372 that is telescopically received within first portion 320 and axially moveable relative thereto. Hollow guidewire 320 is fixed at the distal end thereof to second portion 372, and is received within the lumen of first portion 370 via an aperture 374. A movable, temperature sensing transducer as described hereinabove is situated within guidewire 320. By extending and retracting second portion 372 relative to first portion 370, the pitch and outer diameter of loops 326 of guidewire 320 can be adjusted for optimal contact with the inner wall of hollow body organ 312.

Although the present invention has been described in detail in terms of preferred embodiments, no limitation on the scope of the invention is intended. The scope of the subject matter in which an exclusive right is claimed is defined in the appended claims.

I claim:

1. A method for sensing the temperature profile of a hollow body organ, comprising the steps of:
providing a catheter;
providing a hollow, self-expanding guidewire that expands when unconstrained into a configuration including a plurality of helical loops of greater diameter than the catheter;
providing a temperature sensor disposable within the lumen of the guidewire and moveable longitudinally therein;
contracting the guidewire elastically and constraining the guidewire within the lumen of the catheter;
advancing the catheter and guidewire to a region of interest in a hollow body organ;
withdrawing the catheter while securing the guidewire against substantial longitudinal movement relative to the hollow body organ, whereby the guidewire self-expands into loops in contact with the hollow body organ;
moving the temperature probe through the lumen of the guidewire; and
sensing the temperature of the hollow body organ at multiple locations.

2. The method of claim 1 wherein the temperature probe is advanced together with the catheter and guidewire to the region of interest.

3. The method of claim 1, wherein the guidewire comprises a tubular helix.

4. The method of claim 1, wherein the guidewire comprises a material having martensitic transformation properties.

5. The method of claim 4, wherein the guidewire comprises nitinol.

6. The method of claim 1, wherein the guidewire comprises an elastic material.

7. The method of claim 6, wherein the guidewire comprises spring steel.

8. The method of claim 1, wherein the temperature sensor comprises a thermocouple.

9. The method of claim 8, wherein the temperature sensor comprises one leg of the thermocouple and the guidewire comprises another leg of the thermocouple.

10. The method of claim 1, wherein the temperature sensor comprises a thermistor.

11. The method of claim 1, wherein the temperature sensor comprises a thermochromic material.

12. The method of claim 11, wherein the thermochromic material is in thermal contact with the lumen of the guidewire.

13. The method of claim 12, wherein the temperature sensor further includes an optical probe for sensing the color of the thermochromic material.

14. The method of claim 13, wherein the optical probe includes an illumination device for illuminating a region of interest of the guidewire.

15. The method of claim 14, wherein the optical probe includes a sensing device for sensing reflected radiation from the thermochromic material.

16. The method of claim 15, wherein the reflected radiation is in the visible spectrum.

17. The method of claim 15, wherein the reflected radiation is in the infrared spectrum.

18. The method of claim 15, wherein the reflected radiation is in the ultraviolet spectrum.
